(19) United States
(12) Patent Application Publication
(10) Pub. No.: US 2022/0135512 A1
(43) Pub. Date: May 5, 2022

(54) LOW TOXICITY ORGANIC TERTIARY AND QUATERNARY AMINES AND USES THEREOF

(71) Applicant: Angus Chemical Company, Buffalo Grove, IL (US)

(72) Inventors: Jordan QUINN, Mundelein, IL (US); G David GREEN, Cary, IL (US)

(73) Assignee: Angus Chemical Company, Buffalo Grove, IL (US)

(21) Appl. No.: 17/603,905

(22) PCT Filed: Apr. 15, 2020
(86) PCT No.: PCT/US2020/028299

§ 371 Date: Oct. 14, 2021

Related U.S. Application Data
(60) Provisional application No. 62/834,928, filed on Apr. 16, 2019.

(51) Int. Cl. C07C 209/68; C07C 211/09; C07C 211/14; C07C 215/10; C07C 215/08; C07C 217/08

(52) U.S. Cl. CPC C07C 209/68; C07C 211/09; C07C 217/08; C07C 215/10; C07C 215/08; C07C 211/14

(57) ABSTRACT

Tertiary amine and quaternary ammonium compounds of Formula I and/or Formula II are provided. The present technology also provides compositions that include one or more of the compounds that may be useful for electronics processing (e.g., semiconductor processing composition), cleaning, stripping, degreasing, or a combination of two or more thereof. The compounds of Formula I and/or Formula II may be useful as a low toxicity substitute for tetramethylammonium hydroxide.

### SUMMARY OF THE INVENTION

In one aspect, the present technology provides a compound of Formula I and/or a compound of Formula II:

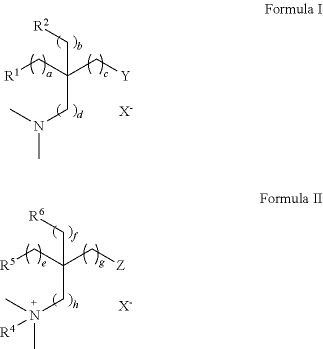

wherein: R¹ and R⁵ are individually H, hydroxyl, or CH₃; R² and R⁶ are individually H, hydroxyl, CH₃, N(CH₃)₂, or ⁺N(CH₃)₃; Y is OR³ or N(R⁸)ᵢ, Z is OR⁷ or N(R¹⁰)ⱼ, wherein R³ and R⁷ are individually H or C₁-C₆ alkyl; R⁸ at each occurrence is individually C₁-C₆ alkyl optionally substituted with one or more hydroxyl groups; and R¹⁰ at each occurrence is individually H or a C₁-C₆ alkyl optionally substituted with one or more hydroxyl groups; R⁴ is H or a C₁-C₆ alkyl group optionally substituted with one or more hydroxyl, amine, or ammonium groups; a, b, d, e, f, and h are individually 0 or 1; c and g are individually 0, 1, or 2; i and j are individually 2 or 3; and X⁻ is an anion and present in the amount equal to the cations present in Formula I or Formula II.

In any embodiment, the compound of Formula I may be a compound of Formula IA and/or the compound of Formula II may be a compound of Formula IIA:

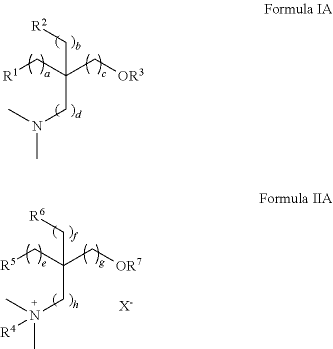

wherein: R¹, R², R⁵, and R⁶ are individually H, hydroxyl, or CH₃; R³ and R⁷ are individually H or C₁-C₆ alkyl; R⁴ is H or a C₁-C₆ alkyl group optionally substituted with one or more hydroxyl groups; a, b, d, e, f and h are individually 0 or 1; c and g are individually 0, 1, or 2; and X⁻ is an anion.

In any embodiment, the compound of Formula I may be a compound of Formula IB and/or the compound of Formula II may be a compound of Formula IIB:

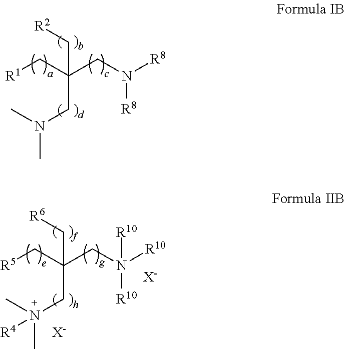

wherein: R¹ and R⁵ are individually H, hydroxyl, or CH₃; R² and R⁶ are individually H, CH₃, N(CH₃)₂, or ⁺N(CH₃)₃; wherein R⁸ at each occurrence is individually C₁-C₆ alkyl optionally substituted with one or more hydroxyl groups; and R¹⁰ at each occurrence is individually H or a C₁-C₆ alkyl optionally substituted with one or more hydroxyl groups; R⁴ is H or a C₁-C₆ alkyl group optionally substituted with one or more hydroxyl, N(CH₃)₂, or ⁺N(CH₃)₃ groups; a, b, d, e, f and h are individually 0 or 1; c and g are individually 0, 1, or 2; and X⁻ is an anion.

The present technology also provides compositions that include a compound of Formula I (or species Formula IA and/or IB), a compound of Formula II (or species Formula IIA and/or IIB), or a combination thereof. In any embodiment, the composition may be useful for electronics processing (e.g., semiconductor processing composition), cleaning, stripping, degreasing, or a combination of two or more thereof. The compound of Formula I (or species Formula IA and/or IB) and/or Formula II (or species Formula IIA and/or IIB) may also be useful as a catalyst, a phase transfer agent, or a combination thereof.

The compound of Formula I (or species Formula IA and/or IB) or Formula II (or species Formula IIA and/or IIB) may also be useful as a substitute for tetramethylammonium hydroxide (“TMAH”)—a highly toxic compound. In any embodiment, the compound of Formula II (or species Formula IIA and/or IIB) may be useful as a substitute for tetramethylammonium hydroxide (“TMAH”)—a highly toxic compound.

In any embodiment, the present technology provides a composition that includes a compound of Formula I (or species Formula IA and/or IB) or Formula II (or species Formula IIA and/or IIB) in place of TMAH. In any embodiment, the present technology provides a composition that includes a compound of Formula II (or species Formula IIA and/or IIB) in place of TMAH.

### DETAILED DESCRIPTION

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

Unless otherwise indicated, numeric ranges, for instance as in “from 2 to 10,” are inclusive of the numbers defining the range (e.g., 2 and 10).

Unless otherwise indicated, ratios, percentages, parts, and the like are by weight.

In general, as used herein, “substituted” refers to an alkyl, alkenyl, alkynyl, aryl, or ether group, as defined below (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group will be substituted with one or more substituents, unless otherwise specified. In some embodiments, a substituted group is substituted with 1, 2, 3, 4, 5, or 6 substituents. Examples of substituent groups include: halogens (i.e., F, Cl, Br, and I); hydroxyls; alkoxy, alkenoxy, alkynoxy, aryloxy, aralkyloxy, heterocyclyloxy, and heterocyclylalkoxy groups; carbonyls (oxo); carboxyls; esters; urethanes; oximes; hydroxylamines; alkoxyamines; aralkoxyamines; thiols; sulfides; sulfoxides; sulfones; sulfonyls; sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; nitriles (i.e., CN); and the like.

As used herein, “alkyl” groups include straight chain and branched alkyl groups having from 1 to about 20 carbon atoms, and typically from 1 to 12 carbons or, in some embodiments, from 1 to 8, 1 to 6, or 1 to 4 carbon atoms. As employed herein, “alkyl groups” include cycloalkyl groups as defined below. Alkyl groups may be substituted or unsubstituted. Examples of straight chain alkyl groups include methyl, ethyl, n-propyl, n-butyl, n-pentyl, n-hexyl, n-heptyl, and n-octyl groups. Examples of branched alkyl groups include, but are not limited to, isopropyl, sec-butyl, t-butyl, neopentyl, and isopentyl groups. Representative substituted alkyl groups may be substituted one or more times with, for example, amino, thio, hydroxy, cyano, alkoxy, and/or halo groups such as F, Cl, Br, and I groups. As used herein the term haloalkyl is an alkyl group having one or more halo groups. In some embodiments, haloalkyl refers to a per-haloalkyl group. In some embodiments, the alkyl group may be substituted. In some embodiments, the alkyl group may be substituted with one or more hydroxyl, amine, or ammonium groups. In other embodiments, the alkyl group may be unsubstituted.

The term “carboxyl” or “carboxylate” as used herein refers to a —C(O)OH group or to its ionized form, —C(O)O—.

The term “carbonyl” as used herein refers to a —C(O)— group.

The term “hydroxyl” as used herein can refer to —OH or its ionized form, —O—.

The term “amine” (or “amino”) as used herein refers to —NR⁶⁵R⁶⁶ groups, wherein R⁶⁵ and R⁶⁶ are independently hydrogen, or a substituted or unsubstituted alkyl, alkenyl, alkynyl, cycloalkyl, aryl, aralkyl, heterocyclylalkyl or heterocyclyl group as defined herein. In some embodiments, the amine is alkylamino, dialkylamino, arylamino, or alkylarylamino. In some embodiments, the amine is an alkylamino or dialkylamino group. In some embodiments, the amine group may be a quaternary amine, which as used herein refers to ammonium groups. In some embodiments, the ammonium includes one, two, or three alkyl groups. The alkyl groups may independently include straight chain and branched alkyl groups having from 1 to about 20 carbon atoms, and typically from 1 to 12 carbons or, in some embodiments, from 1 to 8, 1 to 6, 1 to 4, 1 to 3, or 1 to 2 carbon atoms. In some embodiments, the alkyl group may be a methyl group.

Groups described herein having two or more points of attachment (i.e., divalent, trivalent, or polyvalent) within the compound of the present technology are designated by use of the suffix, “ene.” For example, divalent alkyl groups are alkylene groups, divalent aryl groups are arylene groups, divalent heteroaryl groups are divalent heteroarylene groups, and so forth. Substituted groups having a single point of attachment to the compound of the present technology are not referred to using the “ene” designation.

In one aspect, the present technology provides a compound of Formula I and/or a compound of Formula II:

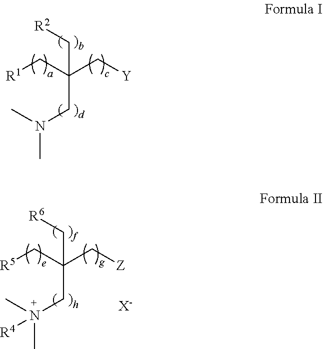

wherein: R¹ and R⁵ are individually H, hydroxyl, or CH₃; R² and R⁶ are individually H, hydroxyl, CH₃, N(CH₃)₂, or ⁺N(CH₃)₃; Y is OR³ or N(R⁸)ᵢ, Z is OR⁷ or N(R¹⁰)ⱼ, wherein R³ and R⁷ are individually H or C₁-C₆ alkyl; R⁸ at each occurrence is individually C₁-C₆ alkyl optionally substituted with one or more hydroxyl groups; and R¹⁰ at each occurrence is individually H or a C₁-C₆ alkyl optionally substituted with one or more hydroxyl groups; R⁴ is H or a C₁-C₆ alkyl group optionally substituted with one or more hydroxyl, amine, or ammonium groups; a, b, d, e, f, and h are individually 0 or 1; c and g are individually 0, 1, or 2; i and j are individually 2 or 3; and X⁻ is an anion and present in the amount equal to the cations present in Formula I or Formula II.

In one embodiment, the compound of Formula I may be a compound of Formula IA and/or the compound of Formula II may be a compound of Formula IIA:

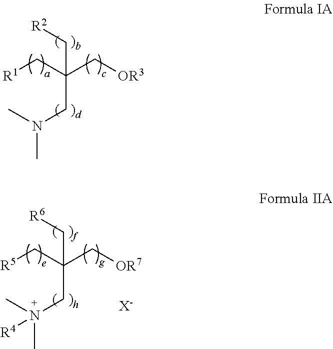

wherein: R¹, R², R⁵, and R⁶ are individually H, hydroxyl, or CH₃; R³ and R⁷ are individually H or C₁-C₆ alkyl; R⁴ is H or a C₁-C₆ alkyl group optionally substituted with one or more hydroxyl groups; a, b, d, e, f and h are individually 0 or 1; c and g are individually 0, 1, or 2; and X⁻ is an anion.

In another embodiment, the compound of Formula I may be a compound of Formula IB and/or the compound of Formula II may be a compound of Formula IIB:

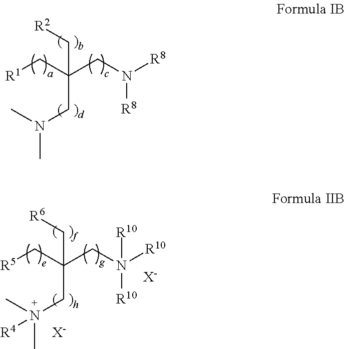

wherein: R¹ and R⁵ are individually H, hydroxyl, or CH₃; R² and R⁶ are individually H, CH₃, N(CH₃)₂, or ⁺N(CH₃)₃; wherein R⁸ at each occurrence is individually C₁-C₆ alkyl optionally substituted with one or more hydroxyl groups; and R¹⁰ at each occurrence is individually H or a C₁-C₆ alkyl optionally substituted with one or more hydroxyl groups; R⁴ is H or a C₁-C₆ alkyl group optionally substituted with one or more hydroxyl, N(CH₃)₂, or ⁺N(CH₃)₃ groups; a, b, d, e, f and h are individually 0 or 1; c and g are individually 0, 1, or 2; and X⁻ is an anion.

In some embodiments, c and g may be 1. In some embodiments, c and g may be 0.

In some embodiments, a and e may be 0. In some embodiments, a and e may be 1.

In some embodiments, b and f may be 0. In some embodiments, b and f may be 1.

In some embodiments, d and h may be 0. In some embodiments, d and h may be 1.

In some embodiments, a, b, c, e, f and g are 1. In some embodiments, a, b, c, e, f and g may be 0.

In some preferred embodiments, a+b+c≥1. In some preferred embodiments, e+f+g≥1. In some preferred embodiments, a+b+c+d≥1. In some preferred embodiments, e+f+g+h≥1. In some preferred embodiments, a+b+c+d≥2. In some preferred embodiments, e+f+g+h≥2. In some preferred embodiments, a+b+c+d≥3. In some preferred embodiments, e+f+g+h≥3.

In some embodiments, a, b, e, and f may be 0; c and g may be 2; and d and h may be 1.

In some embodiments, a, b, e, and f may be 0 and c, d, g, and h may be 1.

In some embodiments, a, b, d, e, f and h may be 1 and c and g may be 0.

In some embodiments, a, b, d, e, f and h may be 0 and c and g may be 1.

In some embodiments, a, b, c, e, f and g may be 0 and d and h may be 1.

In some embodiments, a, d, e, and h may be 0 and b, c, f and g may be 1.

In some embodiments, a, b, d, e, f and h may be 0 and c and g may be 1.

In some embodiments, R¹ and R⁵ may be individually hydroxyl or CH₃. In some embodiments, R¹ and R⁵ may be H or CH₃. In some embodiments, R¹ and R⁵ may be H. In some embodiments, R¹ and R⁵ may be CH₃.

In some embodiments, R³ and R⁷ may be H. In some embodiments, R³ and R⁷ may be individually C₁-C₆ alkyl. In some embodiments, R³ and R⁷ may be individually C₁-C₄ alkyl. In some embodiments, R³ and R⁷ may be individually C₁-C₃ alkyl. In some embodiments, R³ and R⁷ may be individually C₁-C₂ alkyl. In some embodiments, R³ and R⁷ may be CH₃.

In some embodiments, R⁴ may be H or a C₁-C₆ alkyl group optionally substituted with one or more hydroxyl groups. In some embodiments, R⁴ may be a C₁-C₆ alkyl group optionally substituted with one or more hydroxyl groups. In some embodiments, R⁴ may be C₁-C₄ alkyl optionally substituted with one or more hydroxyl groups. In some embodiments, R⁴ may be C₁-C₃ alkyl optionally substituted with one or more hydroxyl groups. In some embodiments, R⁴ may be C₁-C₂ alkyl optionally substituted with one or more hydroxyl groups. In some embodiments, R⁴ may be a methyl group optionally substituted with one or more hydroxyl groups. In some embodiments, R⁴ may be substituted with the one or more hydroxyl groups. In some embodiments, R⁴ may be terminally substituted with the one or more hydroxyl groups. In some embodiments, R⁴ may be substituted with two or more hydroxyl groups. In some embodiments, R⁴ may be terminally substituted with one of the two or more hydroxyl groups. In some embodiments, R⁴ may be a CH₂CH(CH₃)OH group. In some embodiments, R⁴ may be substituted with one or more halide groups. In some embodiments, R⁴ may be substituted with two or more halide groups. In some embodiments, R⁴ may be substituted with one or more hydroxyl groups and one or more halide groups. In some embodiments, R⁴ may be substituted with one or more hydroxyl groups and not with any halide groups. In some embodiments, R⁴ may be substituted with one or more halide groups and not with any hydroxyl groups. In some embodiments, R⁴ may be H or a C₁-C₆ alkyl group optionally substituted with one or more amine or ammonium groups. In some embodiments, R⁴ may be C₁-C₆ alkyl group optionally substituted with one or more amine or ammonium groups. In some embodiments, R⁴ may be C₁-C₄ alkyl optionally substituted with one or more amine or ammonium groups. In some embodiments, R⁴ may be C₁-C₃ alkyl optionally substituted with one or more amine or ammonium groups. In some embodiments, R⁴ may be C₁-C₂ alkyl optionally substituted with one or more amine or ammonium groups. In some embodiments, R⁴ may be a methyl group optionally substituted with one or more amine or ammonium groups. In some embodiments, R⁴ may be an alkyl group substituted with the one or more ⁺N(C₁-C₂ alkyl)₃ groups. In some embodiments, R⁴ may be an alkyl group substituted with the one or more ⁺N(CH₃)₃ groups. In some embodiments, R⁴ may be an alkyl group substituted with the one or more ⁺NH(CH₃)₂ groups. In some embodiments, R⁴ may be an alkyl group substituted with the one or more ⁺NH₂(CH₃) groups. In some embodiments, R⁴ may be an alkyl group substituted with the one or more ⁺NH₃ groups. In some embodiments, R⁴ may be terminally substituted with the one or more ⁺N(CH₃)₃ groups. In some embodiments, R⁴ may be substituted with one ⁺N(CH₃)₃ group. In some embodiments, R⁴ may be a CH₂C(CH₃)₂—N(CH₃)₃⁺ group.

In some embodiments, R² and R⁶ are H, hydroxyl, CH₃, N(CH₃)₂, or ⁺N(CH₃)₃. In some embodiments, R² and R⁶ are H. In some embodiments, R² and R⁶ are hydroxyl. In some embodiments, R² and R⁶ are CH₃. In some embodiments, R² and R⁶ are N(CH₃)₂ or ⁺N(CH₃)₃.

In some embodiments, i is 2. In some embodiments, j is 2 or 3. In some embodiments, j is 3.

In some embodiments, R⁸ and R¹⁰ at each occurrence are individually C₁-C₆ alkyl optionally substituted with one or more hydroxyl groups. In some embodiments, R⁸ and R¹⁰ at each occurrence are individually C₁-C₄ alkyl optionally substituted with one or more hydroxyl groups. In some embodiments, R⁸ and R¹⁰ at each occurrence are individually C₁-C₄ alkyl substituted with one hydroxyl group or CH₃. In some embodiments, R⁸ and R¹⁰ at each occurrence are the same. In some embodiments, R⁸ and R¹⁰ at each occurrence are CH₃. In some embodiments, R⁸ and R¹⁰ at two occurrences are the same. In some embodiments, two R⁸ and two R¹⁰ are CH₃ and the third occurrence of R⁸ and R¹⁰ are a C₁-C₄ alkyl substituted with one hydroxyl group. In some embodiments, one R⁸ and two R¹⁰ are CH₃ and the remaining occurrence of R⁸ and R¹⁰ are a C₁-C₄ alkyl substituted with one hydroxyl group. In some embodiments, R⁸ and R¹⁰ are selected from the group consisting of CH₃ and C(CH₃)₂CH₂OH. In some embodiments, two R⁸ and two R¹⁰ are CH₃ and the third R⁸ and R¹⁰ are a C(CH₃)₂CH₂OH group. In some embodiments, one R⁸ and two R¹⁰ are CH₃ and the remaining R⁸ and R¹⁰ are a C(CH₃)₂CH₂OH group. In some embodiments, R⁸ is CH₃ and C(CH₃)₂CH₂OH and R¹⁰ is CH₃, CH₃, and C(CH₃)₂CH₂OH. In some embodiments, R⁸ and R¹⁰ at each occurrence are CH₃.

In some embodiments, i is 2. In some embodiments, j is 2 or 3. In some embodiments, j is 3.

X⁻ is an anion and present in the amount equal to the cations present in Formula I and/or Formula II. In some embodiments, Formula I may not have any cations and X⁻ is not present as a counter anion. In some embodiments, Formula II may have 1, 2, or 3 cations and X⁻ is present as an anion equal to the amount of cation charges. In some embodiments, X⁻ may be a halide, sulfate, methosulfate, carbonate, carboxylate, phosphate, or hydroxide. In some embodiments, X⁻ may be a halide or hydroxide. In some embodiments, X⁻ may be a hydroxide.

In some embodiments, the composition includes the compound of Formula IA.

In some embodiments, the compound of Formula IA includes

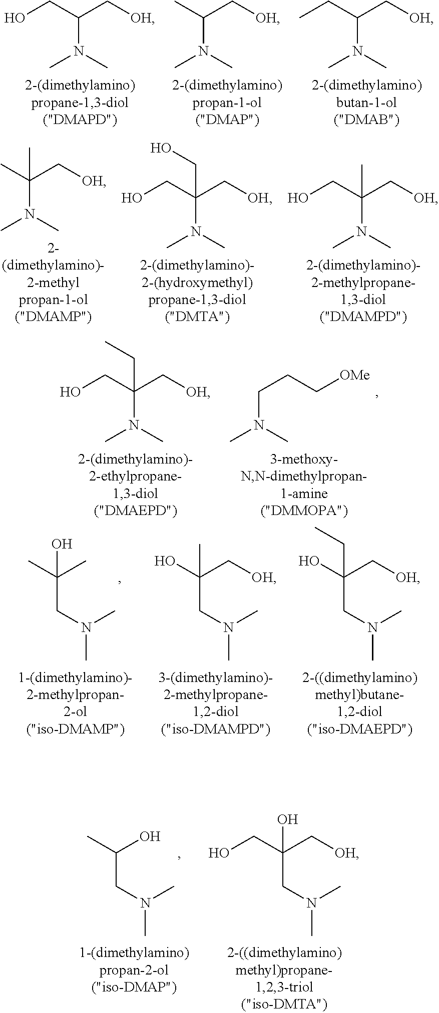

or a combination of two or more thereof.

In some embodiments, the composition the compound of Formula IA includes DMTA, DMAMPD, DMAEPD, DMMOPA, iso-DMTA, iso-DMAMPD, iso-DMAEPD, iso-DMAMP, or a combination of two or more thereof. In some embodiments, the composition the compound of Formula IA includes DMAMPD, iso-DMAMPD, or a combination thereof. In some embodiments, the composition the compound of Formula IA is DMAMPD.

In some preferred embodiments, the composition includes the compound of Formula IIA.

In some embodiments, the compound of Formula IIA includes

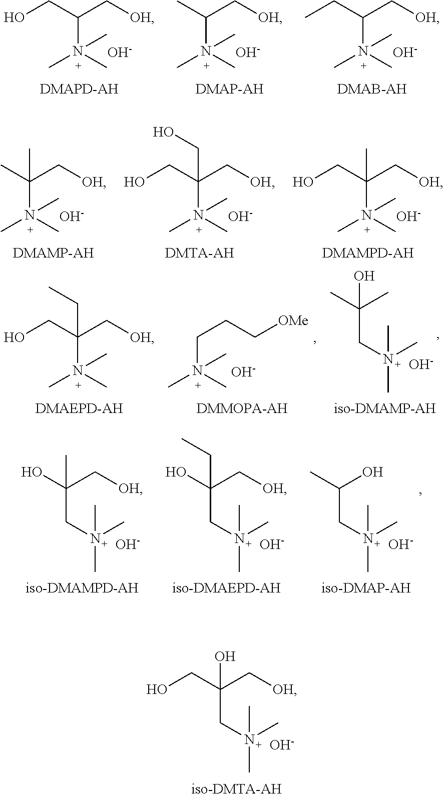

or a combination of two or more thereof.

In some embodiments, the compound of Formula IIA includes DMTA-AH, DMAMPD-AH, DMAEPD-AH, DMMOPA-AH, iso-DMTA-AH, iso-DMAMPD-AH, iso-DMAEPD-AH, iso-DMAMP-AH, or a combination of two or more thereof. In some embodiments, the compound of Formula II includes DMAMP-AH, iso-DMAEPD-AH, or a combination thereof. In some embodiments, the compound of Formula IIA includes DMAMPD-AH, iso-DMAMPD-AH, or a combination thereof. In some embodiments, the compound of Formula IIA includes DMAMPD-AH.

In some preferred embodiments, the composition includes the compound of Formula IB.

In some embodiments, the compound of Formula IB includes

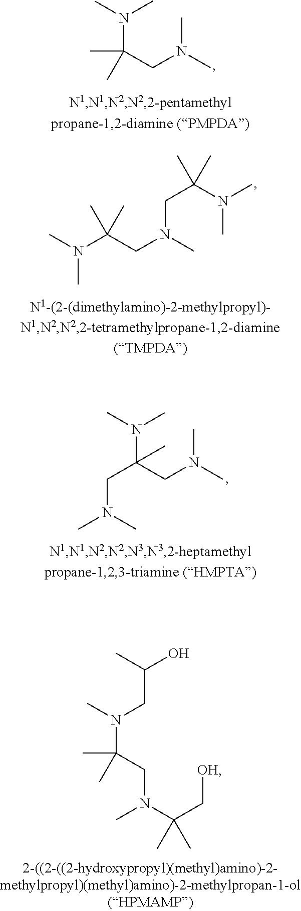

or a combination of two or more thereof.

In some preferred embodiments, the composition includes the compound of Formula IIB.

In some embodiments, the compound of Formula IIB includes

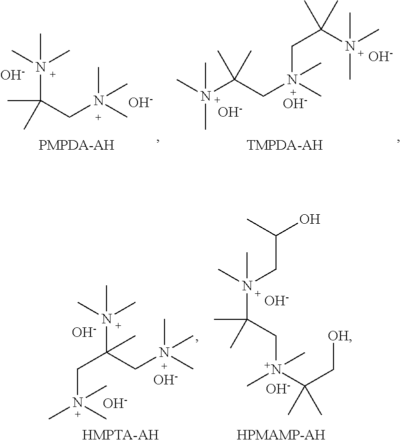

or a combination of two or more thereof.

In some embodiments, the composition has about 0.1 wt % to about 80 wt % of the compound of Formula I and/or Formula II (including Formula IA, IIA, IB, and/or IIB), based on the total composition. In some embodiments, the composition has about 1 wt % to about 50 wt % of the compound of Formula I and/or Formula II (including Formula IA, IIA, IB, and/or IIB), based on the total composition. In some embodiments, the composition has about 0.1 wt % to about 80 wt % of the compound of Formula II (including Formula IIA and/or IIB), based on the total composition. In some embodiments, the composition has about 1 wt % to about 50 wt % of the compound of Formula II (including Formula IIA and/or IIB), based on the total composition.

In some embodiments, the composition may be an electronics processing composition (e.g., semiconductor processing composition), cleaning composition, stripping composition, a degreasing composition, a catalyst, a phase transfer agent, or a combination of two or more thereof. In some embodiments, the electronics processing composition may be a wafer cleaning composition, anisotropic etching composition, photolithography composition, photoresist developing composition, post chemical mechanical planarization cleaning composition, printed circuit board cleaning composition, or a combination of two or more thereof.

In some embodiments, the composition further comprises a stabilizer (e.g., an antioxidant, oxygen scavenger, or reducing agent such dithionite salts, amines, hydroxylamines, sulfites, hydroquinones, hydrides, carboxylic acids, piperazines, formaldehyde, thiourea, borates, butylated hydroxytoluenes, butylated hydroxyanisoles, ascorbic acid, and the like and combinations of two or more thereof).

In some embodiments, the composition further comprises a corrosion inhibitor. In any embodiment the corrosion inhibitor may comprise saccharide, alcohol saccharides, pyrocatechol and/or butylcatechol. Non-limiting examples of saccharide or alcohol saccharides include arabinose, galactose, xylitol, sorbitol, mannitol, mannose, glucose, lactose, maltol, maltose, inositol, xylose, ribose, trehalose, sucrose, fructose and polydextrose).

In some embodiments, the composition further comprises alcohol, pyrocatechol, glycerin and/or glycerin derivative.

In some embodiments, the compound of Formula I and/or the compound of Formula II (including Formula IA, IIA, IB, and/or IIB) is an etching agent. In some embodiments, the compound of Formula II is an etching agent.

In some embodiments, the composition further comprises a hydroxylamine (e.g., hydroxylamine, hydroxylamine sulfate, hydroxylamine chloride, hydroxylamine oxalate, N,N-diethyl hydroxylamine, isopropyl hydroxylamine, dimethyl hydroxylamine hydrochloride and/or hydroxylamine phosphate).

In some embodiments, the composition further comprises an inorganic alkali compound (e.g., sodium hydroxide, potassium hydroxide, ammonia and hydrated hydrazine).

In some embodiments, the composition further comprises water.

In some embodiments, the composition further comprises a hydrotropic component (e.g., cumenesulfonic acid, tetramethylammonium cumenesulfonate, tetramethylammonium xylenesulfonate, tetramethylammonium phenolsulfonate, tetramethylammonium toluenesulfonate, and/or tetramethylammonium benzenesulfonate).

In some embodiments, the composition further comprises a pH adjusting agent, buffering agent, or a combination thereof (e.g, amine, amino alcohol, hydroxylamine, amine oxide, organic hydroxide, phosphate, carbonate, and combinations of two or more thereof).

In some embodiments, the composition further comprises at least one organic solvent or surfactant (e.g., DMSO, an alcohol, ethoxylated alcohol, betaine, alkylsulfonic acid, arylsulfonic acid, or combinations of two or more thereof).

In some embodiments, the composition further comprises a rheology modifier (e.g., polymeric rheology modifier such as polyacrylic acid).

In some embodiments, the composition further comprises a chelating agent (e.g., ethylenediamine tetraacetic acid (“EDTA”)).

In some embodiments, the composition comprises at most about 1000 ppb of a metal selected from the group consisting of Na, Mg, Al, K, Ca, Mn, Fe, Ni, Cu, Zn, Ag, Pd, and Cr.

In some embodiments, the composition has a reduced toxicity compared to a composition comprising the same wt % of tetramethylammonium hydroxide (TMAH), based on rat dermal toxicity.

In some embodiments, the composition may be essentially free of TMAH. As used herein, “essentially free” refers to less than about 5 wt %, less than about 4 wt %, less than about 3 wt %, less than about 2 wt %, less than about 1 wt %, less than about 0.5 wt %, less than about 0.1 wt %, less than about 0.05 wt %, less than about 0.01 wt %, or free of detectable amounts of TMAH.

In another aspect, the present technology provides a process for making the composition comprising a compound of Formula II as provided herein. The process may include: providing a solution comprising a compound of Formula I as recited in any of claims 1-35 and an organic solvent (e.g., isopropyl alcohol); optionally heating the solution to between about 25° C. and about 45° C.; adding a methylating agent (e.g., methyl iodide) to the solution to produce a mixture, wherein the methylating agent is optionally added over a period of about 1 hour; mixing (e.g., stirring) the mixture; optionally cooling the mixture to less than about 20° C. (e.g., 10° C.); and optionally filtering the mixture (e.g., ion exchange resin filter).

In another aspect, the present technology provides a compound of Formula I or a compound of Formula II as provided herein (including Formula IA, IIA, IB, and/or IIB).

In some embodiments, the compound of Formula I includes a compound of Formula IA or Formula IB:

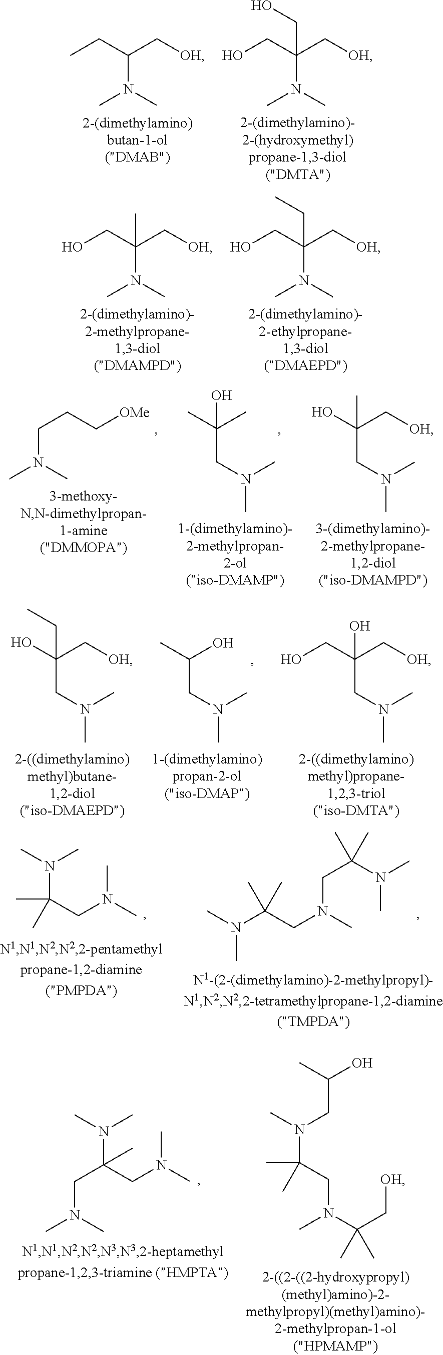

or a combination of two or more thereof. In some embodiments, the compound of Formula I includes DMTA, DMAMPD, DMAEPD, DMMOPA, iso-DMTA, iso-DMAMPD, iso-DMAEPD, iso-DMAMP, PMPDA, TMPDA, HMPTA, HPMAMP, or a combination of two or more thereof. In some embodiments, the compound of Formula I includes DMTA, DMAEPD, DMMOPA, iso-DMTA, iso-DMAMPD, DMAMPD, iso-DMAEPD, iso-DMAMP, PMPDA, TMPDA, HMPTA, HPMAMP, or a combination of two or more thereof. In some embodiments, the compound of Formula I includes DMAMPD, iso-DMAMPD, or a combination thereof. In some embodiments, the compound of Formula I includes DMAMPD.

In some embodiments, the compound of Formula I (including Formulas IA and IB) does not include

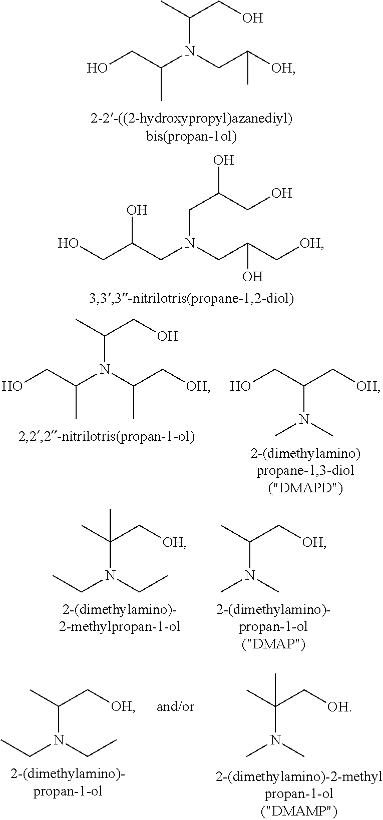

In some embodiments, the compound of Formula II includes a compound of Formula IIA or Formula IIB:

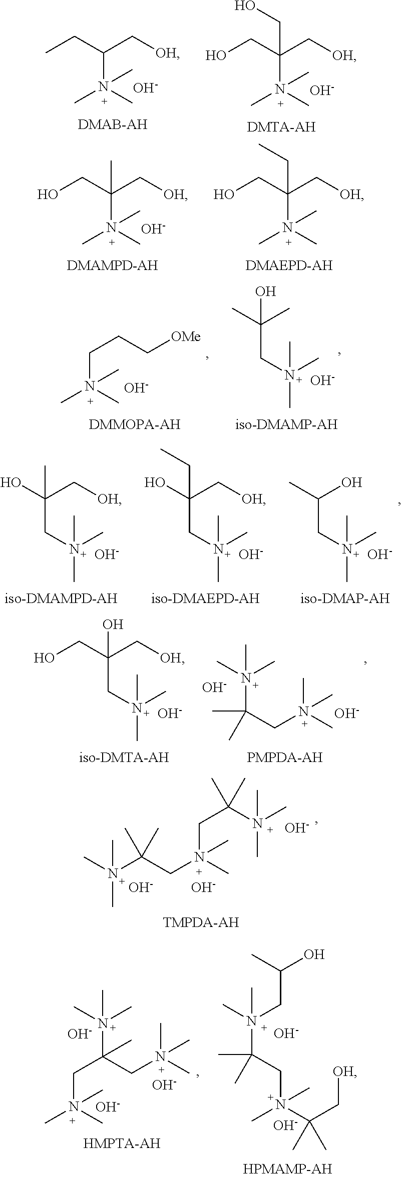

or a combination of two or more thereof. In some embodiments, the compound of Formula II includes DMTA-AH, DMAMPD-AH, DMAEPD-AH, DMMOPA-AH, iso-DMTA-AH, iso-DMAMPD-AH, iso-DMAEPD-AH, iso-DMAMP-AH, PMPDA-AH, TMPDA-AH, HMPTA-AH, HPMAMP-AH, or a combination of two or more thereof. In some embodiments, the compound of Formula II includes DMAMP-AH, iso-DMAEPD-AH, or a combination thereof. In some embodiments, the compound of Formula II includes DMAMPD-AH, iso-DMAMPD-AH, or a combination thereof. In some embodiments, the compound of Formula II includes DMAMPD-AH.

In some embodiments, the compound of Formula II (including Formulas IIA and IIB) does not include

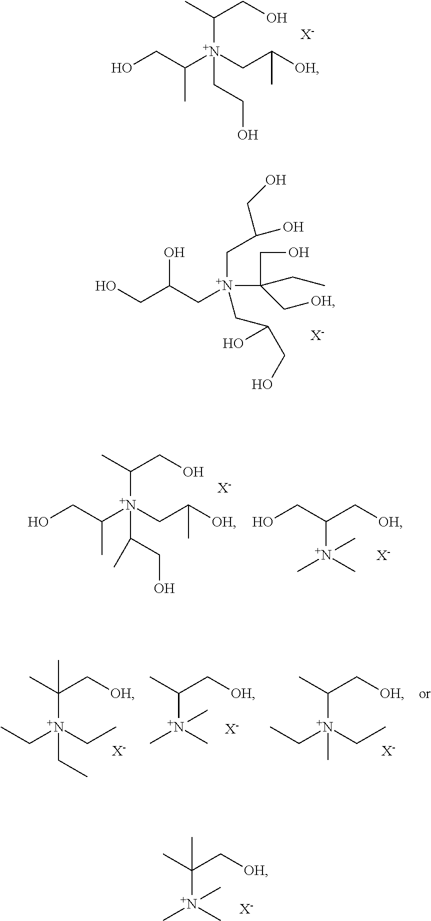

wherein X⁻ is as defined herein.

The present invention, thus generally described, will be understood more readily by reference to the following examples, which are provided by way of illustration and are not intended to be limiting of the present invention.

### EXAMPLES

Example 1. Synthesis of DMAMP-iodide. To a solution of 147 g (1.00 mol) of DMAMP-80 (ANGUS Chemical Company) and 580 g of isopropyl alcohol (99%, VWR International) was added 145 g (1.02 mol) of methyl iodide (99%, Aldrich Chemical Company) in portions over 60 minutes while maintaining the internal temperature between 25° C. and 45° C. Upon complete addition, the mixture was stirred at ambient temperature for 3 hours and then cooled at 10° C. for 1 hour. The mixture was pressure filtered under nitrogen. The filter cake was washed with 500 g of isopropyl alcohol and then dried under vacuum to constant weight. The product was a white solid weighing 242 g (94% yield).

Example 2. Synthesis of DMAMP-AH. DMAMP-iodide from Example 1 was subjected to ion exchange chromatography. A 3×60 cm glass column having a bed volume of 350 mL was used. The column was loaded with AMBERLITE™ IRA410 C₁ (Fisher Scientific) and deionized water. The column was prepared by rinsing the resin with 2 bed volumes of deionized water at a rate of 20 mL/min. The resin was activated by pumping 2 bed volumes of 8 wt % aqueous sodium hydroxide at a rate of 2 mL/min. The resin was rinsed with 6 bed volumes of deionized water to produce neutral eluate. 100 g of DMAMP-iodide was dissolved in 260 g of deionized water and loaded onto the column at 2 mL/min. The eluate was collected when the pH was above 10 (determined using ColorpHast pH strips, EMD/Merck). Approximately 3 bed volumes were collected. The product was concentrated at below 20 mmHg and below 35° C. to give 125 mL of a yellow liquid. The product contained no detectible iodide by peroxide/cellulose test. The product contained 44 wt % solid by HCl titration with bromocresol purple indicator.

Example 3. Synthesis of 3-methoxy-N,N,N-trimethylpropan-1-ammonium hydroxide. To a solution of 117.2 g (1.00 mol) of N,N-dimethyl-3-methoxypropylamine (DMMOPA) (99%, ANGUS Chemical Company) and 520 g of isopropyl alcohol (99%, VWR International) was added 145 g (1.02 mol) of methyl iodide (99%, Aldrich Chemical Company) in portions over 90 minutes while maintaining the internal temperature between 25° C. and 45° C. Upon complete addition, the mixture was stirred at ambient temperature for 4 hours. The mixture was pressure filtered under nitrogen. The filter cake was washed twice with 200 g of isopropyl alcohol and then dried under vacuum to constant weight to provide 3-methoxy-N,N,N-trimethylpropan-1-ammonium iodide. The 3-methoxy-N,N,N-trimethylpropan-1-ammonium iodide was a colorless solid weighing 250.0 g (96.9% yield). ¹H NMR (D₂O) was consistent with the assigned structure.

The 3-methoxy-N,N,N-trimethylpropan-1-ammonium iodide was dissolved in a minimum amount of water and converted to 3-methoxy-N,N,N-trimethylpropan-1-ammonium hydroxide following the procedure in Example 2.

Example 4. Synthesis of 1,3-dihydroxy-N,N,N,2-tetramethylpropan-2-ammonium hydroxide. To a solution of 600 g (2.70 mol) of 2-(dimethylamino)-2-methylpropane-1,3-diol (DMAMPD) (60% aqueous, ANGUS Chemical Company) and 1000 g of isopropyl alcohol (99%, VWR International) was added 490 g (3.45 mol) of methyl iodide (99%, Aldrich Chemical Company) in portions over 70 minutes while maintaining the internal temperature between 25° C. and 45° C. Upon complete addition, the mixture was stirred at ambient temperature for 4 hours. The mixture was cooled at 5° C. for 16 hours. The liquid layer was decanted under nitrogen. The solids were washed with 600 g isopropyl alcohol followed by decanting. The solids were dried under vacuum to constant weight to provide 1,3-dihydroxy-N,N,N,2-tetramethylpropan-2-ammonium iodide. The 1,3-dihydroxy-N,N,N,2-tetramethylpropan-2-ammonium iodide was a colorless solid weighing 562 g (75.9% yield). ¹H NMR (D₂O) was consistent with the assigned structure.

The 1,3-dihydroxy-N,N,N,2-tetramethylpropan-2-ammonium iodide was dissolved in a minimum amount of water and converted to 1,3-dihydroxy-N,N,N,2-tetramethylpropan-2-ammonium hydroxide following the procedure in Example 2.

Example 5. Synthesis of 1,3-dihydroxy-N,N,N,2-tetramethylpropan-2-ammonium hydroxide. To a solution of 212 g (0.955 mol) of 2-(dimethylamino)-2-methylpropane-1,3-diol (DMAMPD) (60% aqueous, ANGUS Chemical Company) was added 70 g (1.4 mol) of methyl chloride (99.5+%, Aldrich Chemical Company) in a 1 L Parr reactor. The mixture was heated to 60° C. wherein the pressure was 114 psi. The mixture was agitated for 4 hours at 60° C. after which time the amine was totally consumed as determined by colorimetric titration with hydrochloric acid. The reactor was vented to atmospheric pressure and then pressure purged three times with 20 psi nitrogen. The mixture was cooled to room temperature to provide 1,3-dihydroxy-N,N,N,2-tetramethylpropan-2-ammonium chloride as a pale yellow liquid. ¹H NMR (D₂O) was consistent with the assigned structure.

The 1,3-dihydroxy-N,N,N,2-tetramethylpropan-2-ammonium chloride was dissolved in a minimum amount of water and converted to 1,3-dihydroxy-N,N,N,2-tetramethylpropan-2-ammonium hydroxide following the procedure in Example 2.

Example 6. Synthesis of 2-hydroxy-2-(hydroxymethyl)-N,N,N-trimethylbutan-1-ammonium hydroxide. To a solution of 100 g (0.68 mol) of 2-((dimethylamino)methyl)butane-1,2-diol (iso-DMAEPD) (ANGUS Chemical Company) and 100 g of isopropyl alcohol (99%, VWR International) was added 100 g (0.70 mol) of methyl iodide (99%, Aldrich Chemical Company) in portions over 30 minutes while maintaining the internal temperature between 25° C. and 45° C. Upon complete addition, the mixture was stirred at ambient temperature for 2 hours. The mixture was concentrated to dryness under vacuum at 40° C. The resulting oil solidified during 4 hours under vacuum. The resulting solid was triturated with 100 g of isopropyl alcohol and then filtered under nitrogen. The solids were dried under vacuum to constant weight to provide 2-hydroxy-2-(hydroxymethyl)-N,N,N-trimethylbutan-1-ammonium iodide. The 2-hydroxy-2-(hydroxymethyl)-N,N,N-trimethylbutan-1-ammonium iodide was a colorless solid weighing 164 g (82% yield). ¹H NMR (D₂O) was consistent with the assigned structure.

The 2-hydroxy-2-(hydroxymethyl)-N,N,N-trimethylbutan-1-ammonium iodide was dissolved in a minimum amount of water and converted to 2-hydroxy-2-(hydroxymethyl)-N,N,N-trimethylbutan-1-ammonium hydroxide following the procedure in Example 2.

### Illustrative Embodiments

Paragraph 1. A compound of Formula I or Formula II:

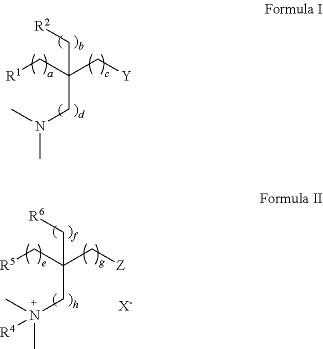

wherein: R¹ and R⁵ are individually H, hydroxyl, or CH₃; R² and R⁶ are individually H, hydroxyl, CH₃, N(CH₃)₂, or ⁺N(CH₃)₃; Y is OR³ or N(R⁸)ᵢ, Z is OR⁷ or N(R¹⁰)ⱼ, wherein: R³ and R⁷ are individually H or C₁-C₆ alkyl; R⁸ at each occurrence is individually C₁-C₆ alkyl optionally substituted with one or more hydroxyl groups; and R¹⁰ at each occurrence is individually C₁-C₆ alkyl optionally substituted with one or more hydroxyl groups; R⁴ is a H or C₁-C₆ alkyl group optionally substituted with one or more hydroxyl, amine, or ammonium groups; a, b, d, e, f and h are individually 0 or 1; c and g are individually 0, 1, or 2; i and j are individually 2 or 3; and X⁻ is an anion and present in the amount equal to the cations present in Formula I or Formula II.

Paragraph 2. The compound of paragraph 1, wherein the compound of Formula I is a compound of Formula IA and/or the compound of Formula II is a compound of Formula IIA:

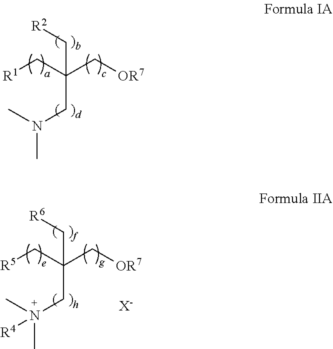

wherein: R¹, R², R⁵, and R⁶ are individually H, hydroxyl, or CH₃; R³ and R⁷ are individually H or C₁-C₆ alkyl; R⁴ is a H or C₁-C₆ alkyl group optionally substituted with one or more hydroxyl groups; a, b, d, e, f and h are individually 0 or 1; c and g are individually 0, 1, or 2; and X⁻ is the anion.

Paragraph 3. The compound of paragraph 1, wherein the compound of Formula I is a compound of Formula IB and/or the compound of Formula II is a compound of Formula IIB:

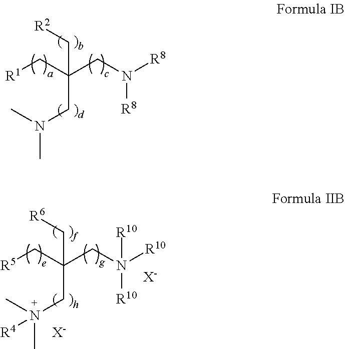

wherein: R¹ and R⁵ are individually H, hydroxyl, or CH₃; R² and R⁶ are individually H, CH₃, N(CH₃)₂, or ⁺N(CH₃)₃; wherein R⁸ at each occurrence is individually C₁-C₆ alkyl optionally substituted with one or more hydroxyl groups; and R¹⁰ at each occurrence is individually C₁-C₆ alkyl optionally substituted with one or more hydroxyl groups; R⁴ is a H or C₁-C₆ alkyl group optionally substituted with one or more hydroxyl, N(CH₃)₂, or ⁺N(CH₃)₃ groups; a, b, d, e, f, and h are individually 0 or 1; c and g are individually 0, 1, or 2; and X⁻ is the anion.

Paragraph 4. The compound of any one of paragraphs 1-3, wherein c and g are 1.

Paragraph 5. The compound of any one of paragraphs 1-3, wherein c and g are 0.

Paragraph 6. The compound of any one of paragraphs 1-5, wherein a and e are 0.

Paragraph 7. The compound of any one of paragraphs 1-5, wherein a and e are 1.

Paragraph 8. The compound of any one of paragraphs 1-7, wherein b and f are 0.

Paragraph 9. The compound of any one of paragraphs 1-7, wherein b and f are 1.

Paragraph 10. The compound of any one of paragraphs 1-9, wherein d and h are 0.

Paragraph 11. The compound of any one of paragraphs 1-9, wherein d and h are 1.

Paragraph 12. The compound of any one of paragraphs 1-3, wherein a, b, c, e, f and g are 1.

Paragraph 13. The compound of any one of paragraphs 1-3, wherein a, b, c, e, f and g are 0.

Paragraph 14. The compound of any one of paragraphs 1-13, wherein a+b+c+d≥1.

Paragraph 15. The compound of any one of paragraphs 1-14, wherein a+b+c+d≥2.

Paragraph 16. The compound of any one of paragraphs 1-15, wherein e+f+g+h≥1.

Paragraph 17. The compound of any one of paragraphs 1-16, wherein e+f+g+h≥2.

Paragraph 18. The compound of any one of paragraphs 1-3, wherein a, b, e, and f are 0 and c, d, g, and h are 1.

Paragraph 19. The compound of any one of paragraphs 1-3, wherein a, b, d, e, f and h are 1 and c and g are 0 or a, b, d, e, f and h are 0 and c and g are 1.

Paragraph 20. The compound of any one of paragraphs 1-3, wherein a, b, c, e, f and g are 0 and d and h are 1.

Paragraph 21. The compound of any one of paragraphs 1-3, wherein a, d, e, and h are 0 and b, c, f and g are be 1.

Paragraph 22. The compound of any one of paragraphs 1-3, wherein a, b, d, e, f and h are 0 and c and g are 1.

Paragraph 23. The compound of any one of paragraphs 1-3, wherein a, b, e, and f are 0; c and g are 2; and d and h are 1.

Paragraph 24. The compound of any one of paragraphs 1-23, wherein R¹ and R⁵ are individually hydroxyl or CH₃.

Paragraph 25. The compound of any one of paragraphs 1-23, wherein R¹ and R⁵ are H or CH₃.

Paragraph 26. The compound of any one of paragraphs 1-25, wherein R¹ and R⁵ are CH₃.

Paragraph 27. The compound of any one of paragraphs 1-26, wherein R³ and R⁷ are H.

Paragraph 28. The compound of any one of paragraphs 1-26, wherein R³ and R⁷ are individually C₁-C₆ alkyl.

Paragraph 29. The compound of paragraph 28, wherein R³ and R⁷ are individually C₁-C₄ alkyl.

Paragraph 30. The compound of paragraph 28 or paragraph 29, wherein R³ and R⁷ are individually C₁-C₃ alkyl.

Paragraph 31. The compound of any one of paragraphs 28-30, wherein R³ and R⁷ are individually C₁-C₂ alkyl.

Paragraph 32. The compound of any one of paragraphs 28-31, wherein R³ and R⁷ are CH₃.

Paragraph 33. The compound of any one of paragraphs 1-32, wherein R⁴ is a C₁-C₄ alkyl group optionally substituted with one or more hydroxyl groups.

Paragraph 34. The compound of paragraph 33, wherein R⁴ is a C₁-C₃ alkyl group optionally substituted with one or more hydroxyl groups.

Paragraph 35. The compound of paragraph 33 or paragraph 34, wherein R⁴ is a C₁-C₂ alkyl group optionally substituted with one or more hydroxyl groups.

Paragraph 36. The compound of any one of paragraphs 33-35, wherein R⁴ is a methyl group optionally substituted with one or more hydroxyl groups.

Paragraph 37. The compound of any one of paragraphs 1-36, wherein R⁴ is substituted with the one or more hydroxyl groups.

Paragraph 38. The compound of paragraph 37, wherein R⁴ is terminally substituted with the one or more hydroxyl groups.

Paragraph 39. The compound of any one of paragraphs 1-38, wherein R⁴ is substituted with two or more hydroxyl groups.

Paragraph 40. The compound of paragraph 39, wherein R⁴ is terminally substituted with one of the two or more hydroxyl groups.

Paragraph 41. The compound of any one of paragraphs 1-34, wherein R⁴ is CH₂CH(CH₃)OH.

Paragraph 42. The compound of any one of paragraphs 1-32, wherein R⁴ is a C₁-C₄ alkyl group optionally substituted with one or more amine or ammonium groups.

Paragraph 43. The compound of paragraph 42, wherein R⁴ is a C₁-C₃ alkyl group optionally substituted with one or more amine or ammonium groups.

Paragraph 44. The compound of paragraph 42 or paragraph 43, wherein R⁴ is a C₁-C₂ alkyl group optionally substituted with one or more amine or ammonium groups.

Paragraph 45. The compound of any one of paragraphs 42-44, wherein R⁴ is a methyl group optionally substituted with one or more amine or ammonium groups.

Paragraph 46. The compound of any one of paragraphs 1-45, wherein R⁴ is substituted with the one or more amine or ammonium groups.

Paragraph 47. The compound of paragraph 46, wherein R⁴ is terminally substituted with one or more ⁺N(CH₃)₃ groups.

Paragraph 48. The compound of paragraph 46 or paragraph 47, wherein R⁴ is a CH₂C(CH₃)₂—N(CH₃)₃⁺.

Paragraph 49. The compound of any one of paragraphs 1-48, wherein R² and R⁶ are H.

Paragraph 50. The compound of any one of paragraphs 1-48, wherein R² and R⁶ are hydroxyl.

Paragraph 51. The compound of any one of paragraphs 1-48, wherein R² and R⁶ are CH₃.

Paragraph 52. The compound of any one of paragraphs 1-48, wherein R² and R⁶ are N(CH₃)₂ or ⁺N(CH₃)₃.

Paragraph 53. The compound of any one of paragraphs 1-52, wherein i is 2.

Paragraph 54. The compound of any one of paragraphs 1-53, wherein j is 2 or 3.

Paragraph 55. The compound of any one of paragraphs 1-54, wherein j is 3.

Paragraph 56. The compound of any one of paragraphs 1-55, wherein R⁸ and R¹⁰ at each occurrence are individually C₁-C₄ alkyl optionally substituted with one or more hydroxyl groups.

Paragraph 57. The compound of any one of paragraphs 1-55, wherein R⁸ and R¹⁰ at each occurrence are individually C₁-C₄ alkyl substituted with one hydroxyl group or CH₃.

Paragraph 58. The compound of any one of paragraphs 1-57, wherein R⁸ and R¹⁰ at each occurrence are the same.

Paragraph 59. The compound of any one of paragraphs 1-58, wherein R⁸ and R¹⁰ at two occurrences are the same.

Paragraph 60. The compound of any one of paragraphs 1-59, wherein R⁸ and R¹⁰ at each occurrence are CH₃.

Paragraph 61. The compound of paragraph 59, wherein R⁸ and R¹⁰ at two occurrences are CH₃ and at a third occurrence R⁸ and R¹⁰ are a C(CH₃)₂CH₂OH group. The compound of paragraph 59, wherein R⁸ and R¹⁰ are selected from the group consisting of CH₃ and C(CH₃)₂CH₂OH. The compound of paragraph 59, wherein R⁸ is CH₃ and C(CH₃)₂CH₂OH and R¹⁰ is CH₃, CH₃, and C(CH₃)₂CH₂OH.

Paragraph 62. The compound of any one of paragraphs 1-61, wherein X⁻ is a halide, sulfate, methosulfate, carbonate, carboxylate, phosphate, or hydroxide.

Paragraph 63. The compound of any one of paragraphs 1-62, wherein X⁻ is a halide or hydroxide.

Paragraph 64. The compound of any one of paragraphs 1-63, wherein X⁻ is a hydroxide.

Paragraph 65. The compound of any one of paragraphs 1-64, wherein the compound of Formula I is a compound of Formula IA selected from the group consisting of

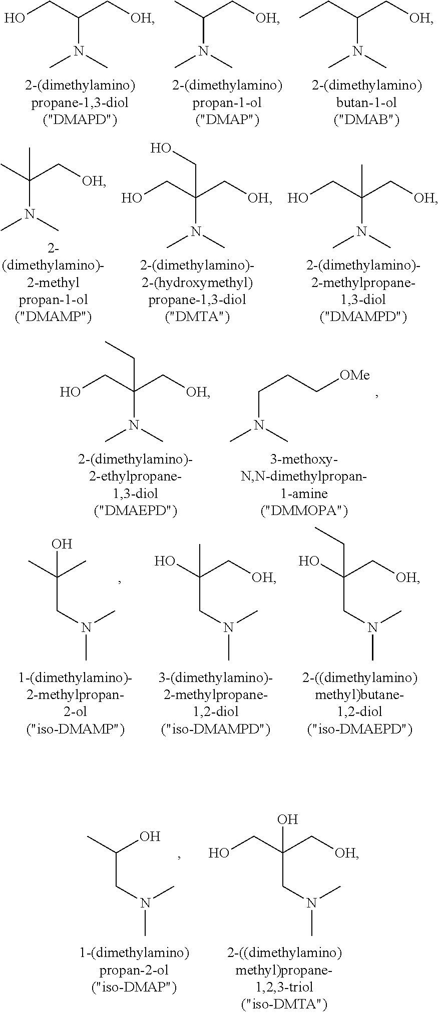

and combinations of two or more thereof.

Paragraph 66. The compound of paragraph 66, wherein the compound of Formula I is a compound of Formula IA selected from the group consisting of DMTA, DMAMPD, DMAEPD, DMMOPA, iso-DMTA, iso-DMAMPD, iso-DMAEPD, iso-DMAMP, and combinations of two or more thereof. The compound of paragraph 66, wherein the compound of Formula I is a compound of Formula IA is DMAMPD.

Paragraph 67. The compound of any one of paragraphs 1-66, wherein the compound of Formula II is a compound of Formula IIA selected from the group consisting of

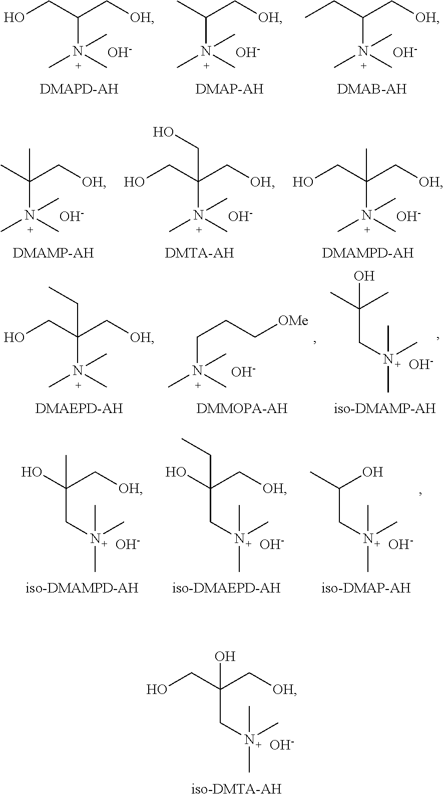

and combinations of two or more thereof.

Paragraph 68. The compound of paragraph 67, wherein the compound of Formula IIA is selected from the group consisting of DMTA-AH, DMAMPD-AH, DMAEPD-AH, DMMOPA-AH, iso-DMTA-AH, iso-DMAMPD-AH, iso-DMAEPD-AH, iso-DMAMP-AH, or a combination of two or more thereof. The composition of paragraph 68, wherein the compound of Formula IIA is DMAMPD-AH.

Paragraph 69. The compound of any one of paragraphs 1-68, wherein the compound of Formula I is a compound of Formula IB selected from the group consisting of

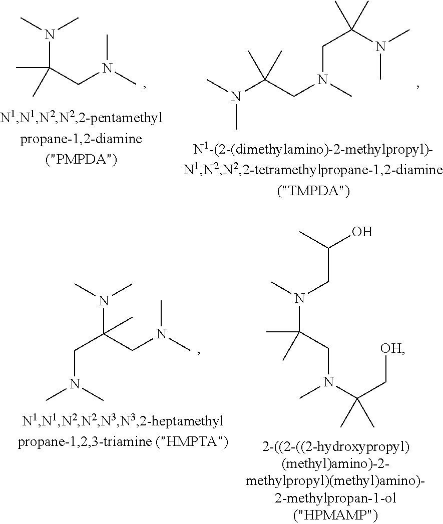

and combinations of two or more thereof.

Paragraph 70. The compound of any one of paragraphs 1-69, wherein the compound of Formula II is a compound of Formula IIB selected from the group consisting of

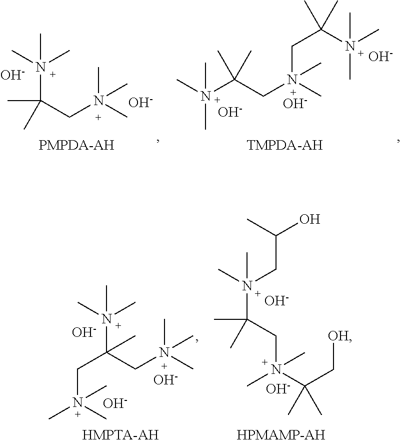

and combinations of two or more thereof.

Paragraph 71. A composition comprising a compound of Formula I (including Formula IA and/or IB), a compound of Formula II (including Formula IIA and/or IIB), or a combination thereof as recited in any one of paragraphs 1-70.

Paragraph 72. The composition of paragraph 71, wherein the composition has about 0.1 wt % to about 80 wt % of the compound of Formula I, the compound of Formula II, or a combination thereof, based on the total composition.

Paragraph 73. The composition of paragraph 71 or paragraph 72, wherein the composition has about 1 wt % to about 50 wt % of the compound of Formula I, the compound of Formula II, or a combination thereof, based on the total composition.

Paragraph 74. The composition of any one of paragraphs 71-73, wherein the composition is an electronics processing composition (e.g., semiconductor processing composition), cleaning composition, stripping composition, a degreasing composition, a catalyst, a phase transfer agent, or a combination of two or more thereof.

Paragraph 75. The composition of 74, wherein the electronics processing composition is a wafer cleaning composition, anisotropic etching composition, photolithography composition, photoresist developing composition, post chemical mechanical planarization cleaning composition, printed circuit board cleaning composition, or a combination of two or more thereof.

Paragraph 76. The composition of any one of paragraphs 71-75, wherein the composition further comprises a stabilizer (e.g., an antioxidant, oxygen scavenger, or reducing agent such dithionite salts, amines, hydroxylamines, sulfites, hydroquinones, hydrides, carboxylic acids, piperazines, formaldehyde, thiourea, borates, butylated hydroxytoluenes, butylated hydroxyanisoles, ascorbic acid, and the like and combinations of two or more thereof).

Paragraph 77. The composition of any one of paragraphs 71-76, wherein the composition further comprises a corrosion inhibitor.

Paragraph 78. The composition of any one of paragraphs 71-77, wherein the composition further comprises alcohol, pyrocatechol, glycerin and/or glycerin derivative.

Paragraph 79. The composition of any one of paragraphs 71-78, wherein the compound of Formula I and/or the compound of Formula II is an etching agent.

Paragraph 80. The composition of any one of paragraphs 71-79, wherein the composition further comprises a hydroxylamine (e.g., hydroxylamine, hydroxylamine sulfate, hydroxylamine chloride, hydroxylamine oxalate, N,N-diethyl hydroxylamine, isopropyl hydroxylamine, dimethyl hydroxylamine hydrochloride and/or hydroxylamine phosphate).

Paragraph 81. The composition of any one of paragraphs 71-80, wherein the composition further comprises an inorganic alkali compound (e.g., sodium hydroxide, potassium hydroxide, ammonia and hydrated hydrazine).

Paragraph 82. The composition of any one of paragraphs 71-81, wherein the composition further comprises water.

Paragraph 83. The composition of any one of paragraphs 71-82, wherein the composition further comprises a hydrotropic component (e.g., cumenesulfonic acid, tetramethylammonium cumenesulfonate, tetramethylammonium xylenesulfonate, tetramethylammonium phenolsulfonate, tetramethylammonium toluenesulfonate, and/or tetramethylammonium benzenesulfonate).

Paragraph 84. The composition of any one of paragraphs 71-83, wherein the composition further comprises a pH adjusting agent, buffering agent, or a combination thereof (e.g, amine, amino alcohol, hydroxylamine, amine oxide, organic hydroxide, phosphate, carbonate, and combinations of two or more thereof).

Paragraph 85. The composition of any one of paragraphs 71-84, wherein the composition further comprises at least one organic solvent or surfactant (e.g., DMSO, an alcohol, ethoxylated alcohol, betaine, alkylsulfonic acid, arylsulfonic acid, or combinations of two or more thereof).

Paragraph 86. The composition of any one of paragraphs 71-85, wherein the composition further comprises a rheology modifier (e.g., polymeric rheology modifier such as polyacrylic acid).

Paragraph 87. The composition of any one of paragraphs 71-86, wherein the composition further comprises a chelating agent (e.g., ethylenediamine tetraacetic acid (“EDTA”)).

Paragraph 88. The composition of any one of paragraphs 71-87, wherein the composition comprises at most about 1000 ppb of a metal selected from the group consisting of Na, Mg, Al, K, Ca, Mn, Fe, Ni, Cu, Zn, Ag, Pd, and Cr.

Paragraph 89. The composition of any one of paragraphs 71-88, wherein the composition has a reduced toxicity compared to a composition comprising the same wt % of tetramethylammonium hydroxide (TMAH), based on rat dermal toxicity.

Paragraph 90. The composition of any one of paragraphs 71-89, wherein the composition is essentially free of TMAH.

Paragraph 91. A process for making a compound of Formula II as recited in any of paragraphs 1-70, the process comprising: providing a solution comprising a compound of Formula I as recited in any of paragraphs 1-70 and an organic solvent (e.g., isopropyl alcohol); optionally heating the solution to between about 25° C. and about 45° C.; adding a methylating agent (e.g., methyl iodide) to the solution to produce a mixture, wherein the methylating agent is optionally added over a period of about 1 hour; mixing the mixture; optionally cooling the mixture to less than about 20° C. (e.g., 10° C.); optionally filtering the mixture (e.g., ion exchange resin filter).

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims.

What is claimed is:

1. A composition comprising a compound of Formula I, a compound of Formula II, or a combination thereof: 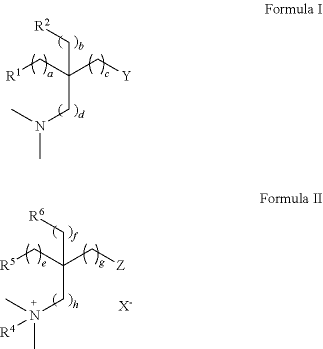
wherein:
R¹ and R⁵ are individually H, hydroxyl, or CH₃;
R² and R⁶ are individually H, hydroxyl, CH₃, N(CH₃)₂, or ⁺N(CH₃)₃;
Y is OR³ or N(R⁸)ᵢ, Z is OR⁷ or N(R¹⁰)ⱼ,
wherein:
R³ and R⁷ are individually H or C₁-C₆ alkyl; and
R⁸ at each occurrence is individually C₁-C₆ alkyl optionally substituted with one or more hydroxyl groups;
R¹⁰ at each occurrence is individually H or a C₁-C₆ alkyl optionally substituted with one or more hydroxyl groups;
R⁴ is H or a C₁-C₆ alkyl group optionally substituted with one or more hydroxyl, amine, or ammonium groups;
a, b, d, e, f, and h are individually 0 or 1;
c and g are individually 0, 1, or 2;
i and j are individually 2 or 3; and
X⁻ is an anion and present in the amount equal to the cations present in Formula I or Formula II.

2. The composition of claim 1, wherein the compound of Formula I is a compound of Formula IA and/or the compound of Formula II is a compound of Formula IIA: 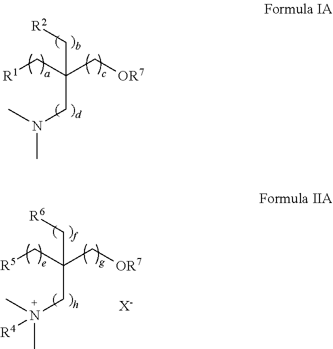
wherein:
R¹, R², R⁵, and R⁶ are individually H, hydroxyl, or CH₃;
R³ and R⁷ are individually H or C₁-C₆ alkyl;
R⁴ is H or a C₁-C₆ alkyl group optionally substituted with one or more hydroxyl groups;
a, b, d, e, f, and h are individually 0 or 1;
c and g are individually 0, 1, or 2; and
X⁻ is the anion.

3. The composition of claim 1, wherein the compound of Formula I is a compound of Formula IB and/or the compound of Formula II is a compound of Formula IIB: 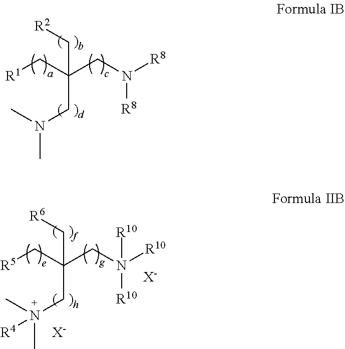
wherein:
R¹ and R⁵ are individually H, hydroxyl, or CH₃;
R² and R⁶ are individually H, CH₃, N(CH₃)₂, or ⁺N(CH₃)₃;
wherein:
R⁸ at each occurrence is individually C₁-C₆ alkyl optionally substituted with one or more hydroxyl groups; and
R¹⁰ at each occurrence is individually H or a C₁-C₆ alkyl optionally substituted with one or more hydroxyl groups;
R⁴ is C₁-C₆ alkyl optionally substituted with one or more hydroxyl, N(CH₃)₂, or ⁺N(CH₃)₃ groups;
a, b, d, e, f, and h are individually 0 or 1;
c and g are individually 0, 1, or 2; and
X⁻ is the anion.

4. The composition of any one of claims 1-3, wherein a+b+c+d≥1 and e+f+g+h≥1.

5. The composition of any one of claims 1-4, wherein a+b+c+d≥2 and e+f+g+h≥2.

6. The composition of any one of claims 1-5, wherein R¹ and R⁵ are individually hydroxyl or CH₃.

7. The composition of any one of claims 1-5, wherein R¹ and R⁵ are H or CH₃.

8. The composition of any one of claims 1-7, wherein R³ and R⁷ are H.

9. The composition of any one of claims 1-7, wherein R³ and R⁷ are individually C₁-C₆ alkyl.

10. The composition of claim 9, wherein R³ and R⁷ are CH₃.

11. The composition of any one of claims 1-10, wherein R⁴ is C₁-C₄ alkyl optionally substituted with one or more hydroxyl groups.

12. The composition of claim 11, wherein R⁴ is a methyl group optionally substituted with one or more hydroxyl groups.

13. The composition of claim 11 or claim 12, wherein R⁴ is terminally substituted with the one or more hydroxyl groups.

14. The composition of any one of claims 1-13, wherein R⁴ is substituted with two or more hydroxyl groups.

15. The composition of any one of claims 1-13, wherein R⁴ is CH₂CH(CH₃)OH.

16. The composition of any one of claims 1-10, wherein R⁴ is C₁-C₄ alkyl optionally substituted with one or more amine or ammonium groups.

17. The composition of claim 16, wherein R⁴ is a methyl group optionally substituted with one or more amine or ammonium groups.

18. The composition of claim 16 or claim 17, wherein R⁴ is terminally substituted with one or more ⁺N(CH₃)₃ groups.

19. The composition of any one of claims 1-10 or 16, wherein R⁴ is a CH₂C(CH₃)₂—N(CH₃)₃⁺.

20. The composition of any one of claims 1-19, wherein R² and R⁶ are H.

21. The composition of any one of claims 1-19, wherein R² and R⁶ are hydroxyl.

22. The composition of any one of claims 1-19, wherein R² and R⁶ are CH₃.

23. The composition of any one of claims 1-19, wherein R² and R⁶ are N(CH₃)₂ or ⁺N(CH₃)₃.

24. The composition of any one of claims 1-23, wherein R⁸ and R¹⁰ at each occurrence are individually C₁-C₆ alkyl optionally substituted with one or more hydroxyl groups.

25. The composition of any one of claims 1-24, wherein R⁸ and R¹⁰ at each occurrence are CH₃.

26. The composition of any one of claims 1-24, wherein R⁸ and R¹⁰ are selected from the group consisting of CH₃ and C(CH₃)₂CH₂OH.

27. The composition of any one of claims 1-26, wherein X⁻ is a halide, sulfate, methosulfate, carbonate, carboxylate, phosphate, or hydroxide.

28. The composition of any one of claims 1-27, wherein X⁻ is a hydroxide.

29. The composition of any one of claims 1-28, wherein the compound of Formula I is a compound of Formula IA selected from the group consisting of 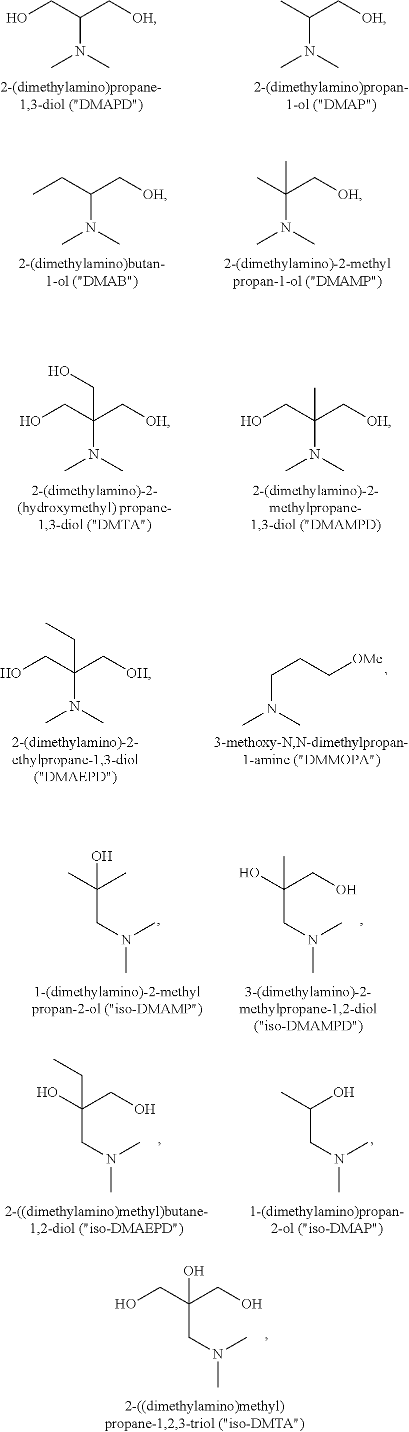
and combinations of two or more thereof.

30. The composition of claim 29, wherein the compound of Formula I is a compound of Formula IA selected from the group consisting of DMAMPD, iso-DMAMPD, and combinations thereof.

31. The composition of any one of claims 1-30, wherein the compound of Formula II is a compound of Formula IIA selected from the group consisting of 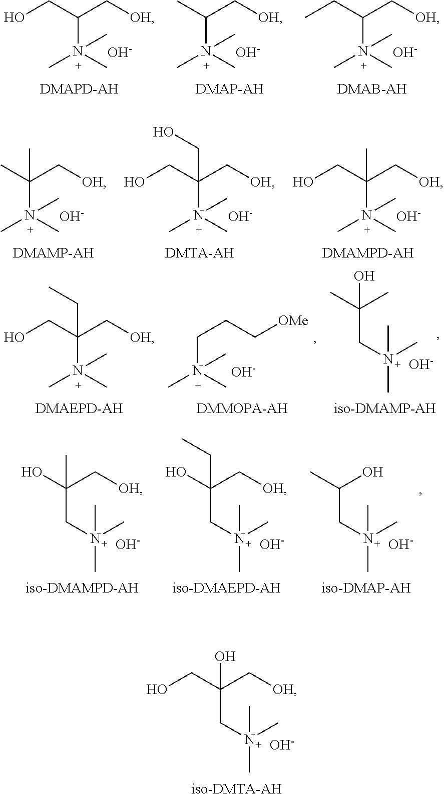
and combinations of two or more thereof.

32. The composition of claim 31, wherein the compound of Formula II is a compound of Formula IIA that is DMAMPD-AH.

33. The composition of any one of claims 1-32, wherein the compound of Formula I is a compound of Formula I selected from the group consisting of 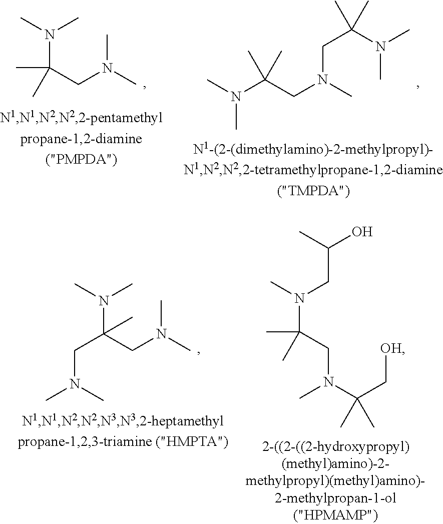
and combinations of two or more thereof.

34. The composition of any one of claims 1-33, wherein the compound of Formula II is a compound of Formula IIB selected from the group consisting of 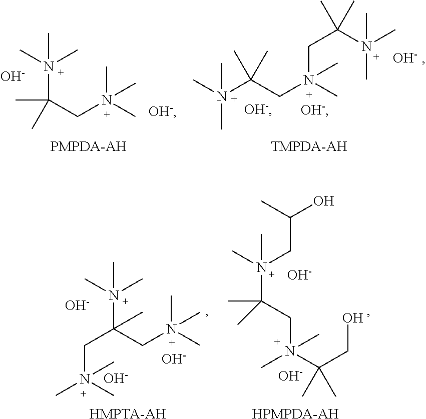
and combinations of two or more thereof.

35. The composition of any one of claims 1-34, wherein the composition has about 0.1 wt % to about 80 wt % of the compound of Formula I, the compound of Formula II, or a combination thereof, based on the total composition.

36. The composition of any one of claims 1-35, wherein the composition has about 1 wt % to about 50 wt % of the compound of Formula I, the compound of Formula II, or a combination thereof, based on the total composition.

37. The composition of any one of claims 1-36, wherein the composition is an electronics processing composition (e.g., semiconductor processing composition), cleaning composition, stripping composition, a degreasing composition, a catalyst, a phase transfer agent, or a combination of two or more thereof.

38. The composition of 37, wherein the electronics processing composition is a wafer cleaning composition, anisotropic etching composition, photolithography composition, photoresist developing composition, post chemical mechanical planarization cleaning composition, printed circuit board cleaning composition, or a combination of two or more thereof.

39. The composition of any one of claims 1-38 further comprising a stabilizer (e.g., an antioxidant, oxygen scavenger, or reducing agent such dithionite salts, amines, hydroxylamines, sulfites, hydroquinones, hydrides, carboxylic acids, piperazines, formaldehyde, thiourea, borates, butylated hydroxytoluenes, butylated hydroxyanisoles, ascorbic acid, and the like and combinations of two or more thereof).

40. The composition of any one of claims 1-39 further comprising a corrosion inhibitor.

41. The composition of any one of claims 1-40 further comprising alcohol, pyrocatechol, glycerin and/or glycerin derivative.

42. The composition of any one of claims 1-41, wherein the compound of Formula I and/or the compound of Formula II is an etching agent.

43. The composition of any one of claims 1-42 further comprising a hydroxylamine (e.g., hydroxylamine, hydroxylamine sulfate, hydroxylamine chloride, hydroxylamine oxalate, N,N-diethyl hydroxylamine, isopropyl hydroxylamine, dimethyl hydroxylamine hydrochloride and/or hydroxylamine phosphate).

44. The composition of any one of claims 1-43 further comprising an inorganic alkali compound.

45. The composition of any one of claims 1-44 further comprising water.

46. The composition of any one of claims 1-45 further comprising a hydrotropic component.

47. The composition of any one of claims 1-46 further comprising a pH adjusting agent, buffering agent, or a combination thereof.

48. The composition of any one of claims 1-47 further comprising at least one organic solvent or surfactant (e.g., DMSO, an alcohol, ethoxylated alcohol, betaine, alkylsulfonic acid, arylsulfonic acid, or combinations of two or more thereof).

49. The composition of any one of claims 1-48 further comprising a rheology modifier (e.g., polymeric rheology modifier such as polyacrylic acid).

50. The composition of any one of claims 1-49 further comprising a chelating agent (e.g., ethylenediamine tetraacetic acid (“EDTA”)).

51. The composition of any one of claims 1-50, wherein the composition comprises at most about 1000 ppb of a metal selected from the group consisting of Na, Mg, Al, K, Ca, Mn, Fe, Ni, Cu, Zn, Ag, Pd, and Cr.

52. The composition of any one of claims 1-51, wherein the composition has a reduced toxicity compared to a composition comprising the same wt % of tetramethylammonium hydroxide (TMAH), based on rat dermal toxicity.

53. The composition of any one of claims 1-52, wherein the composition is essentially free of TMAH.

54. A compound of Formula I or a compound of Formula II as recited in any one of claims 1-34.

55. A process for making the compound of Formula II as recited in claim 54, the process comprising:
providing a solution comprising a compound of Formula I as recited in any of claims 1-34 and an organic solvent;
optionally heating the solution to between about 25° C. and about 45° C.;
adding a methylating agent to the solution to produce a mixture, wherein the methylating agent is optionally added over a period of about 1 hour;
mixing the mixture;
optionally cooling the mixture to less than about 20° C.;
optionally filtering the mixture.
